(19) United States
(12) Patent Application Publication
(10) Pub. No.: US 2003/0211624 A1
(43) Pub. Date: Nov. 13, 2003

(54) Proteomic methods for diagnosis and monitoring of breast cancer

(75) Inventors: Ira L. Goldknopf, The Woodlands, TX (US); Helen R. Park, The Woodlands, TX (US); Herbert A. Fritsche, Houston, TX (US); Henry M. Kuerer, Houston, TX (US)

Correspondence Address: ELIZABETH R. HALL, 1722 MARYLAND STREET, HOUSTON, TX, 77006, US

(73) Assignee: Board of Regents, The University of Texas System

(21) Appl. No.: 10/301,512

(22) Filed: Nov. 20, 2002

Related U.S. Application Data
(60) Provisional application No. 60/331,815, filed on Nov. 20, 2001.
(60) Provisional application No. 60/375,032, filed on Apr. 24, 2002.

(51) Int. Cl. G01N 33/00

(52) U.S. Cl. 436/86; 436/173

(57) ABSTRACT

A method of diagnosing a cancerous condition in a female includes analyzing ductal secretions from each breast of a female subject in which differences in the protein profiles of the two samples are indicative of a cancerous condition. Protein samples are preferable analyzed by two dimensional gel electrophoresis or by microarrays. Identification of cancer markers and monitoring progression and treatment of cancers, and especially breast cancer are also dislcosed.

### CROSS-REFERENCE TO RELATED APPLICATIONS

This application claims benefit of the priority of U.S. Provisional Application No. 60/331,815, filed Nov. 20, 2001 and U.S. Provisional Application No. 60/375,032, filed Apr. 24, 2002, both of which are incorporated herein in their entirety by reference.

### STATEMENT REGARDING FEDERALLY SPONSORED RESEARCH OR DEVELOPMENT

N/A

### BACKGROUND OF THE INVENTION

Attempts have been made in the art to use nipple aspirate fluid to detect and identify breast cancer markers (Liu et al., The Lancet 356:567, 2000; Wrensch et al., Journal of the National Caner Institute 93:1791, 2001; Sauter et al., British Journal of Cancer 85:1952, 2001). However, such studies, while identifying certain markers, are limited by the lack of a baseline state to which to compare the nipple aspirate from the suspected cancerous breast.

### SUMMARY

The present disclosure addresses the problems of the prior art by comparing the two breasts of the same subject in order to diagnose or monitor a cancerous state in one or both breasts. In this way, the overall protein pattern involving more than 1500 proteins in some cases may be analyzed in a single matched pair of samples, one from each breast of the same individual. In addition, by serving as her own control, the subject's hormonal state is reflected in both breasts, removing much of the uncertainty in prior methods.

The present disclosure may be described in certain embodiments as a method for detecting a neoplasm in the breast of a subject, the method including (a) obtaining breast ductal secretions from each breast of the subject; (b) subjecting the proteins from the ductal secretions of each breast to 2D gel electrophoresis to obtain a separate protein pattern for the ductal secretions of each breast; and (c) detecting differences in the protein pattern of one of the breasts compared to the protein pattern of the other breast of the same subject; where a significant difference in the protein pattern of the two breasts is indicative of the presence of a neoplasm in one or both of the breasts.

In preferred embodiments the proteins are separated in one dimension of the two dimensional (2D) gel according to the isoelectric point of the proteins, and in further preferred embodiments, the proteins are separated in one dimension of the two dimensional (2D) gel according to molecular weight. Ductal secretions may be obtained by any known method and are preferable obtained by nipple aspiration, or alternatively by ductal lavage.

In the practice of preferred embodiments, a significant difference in the total number of proteins detected in the protein pattern of one breast of a subject relative to the protein pattern of the other breast of the same subject is indicative of a neoplasm in one or both of the breasts, and in certain embodiments, a significant difference in quantity of one or more proteins in the protein pattern of one breast of a subject relative to the protein pattern of the other breast of the same subject is indicative of a neoplasm in one or both of the breasts. As is well known in the art, a significant difference in protein profiles between the two breasts is a statistical decision based on a population of know unilateral cancer patients and a population of normal subjects. When difference data for the two populations is analyzed with a receiver operating characteristic (ROC) curve, a threshhold number of difference spots is determined to be statistically significant as an indicator of possible disease. As such, some diagnoses may be borderline, or unclear and may simply indicate that further diagnosis is needed. It is understood that the neoplasm may be, but is not limited to cancer, a benign condition, a pre-cancerous condition, an early cancerous condition detectable or not yet detectable by mammography, ductal carcinoma in situ, primary invasive ductal carcinoma or metastatic breast cancer.

It is a further aspect of the disclosure that in certain preferred embodiments, one may isolate one or more proteins that are quantitatively or qualitatively different in the aspirate of a cancerous breast versus a non-cancerous breast of the same subject, and further that the mass fingerprint, peptide mass fingerprint and/or amino acid sequence of the isolated protein may be determined. The detected proteins may include one or proteins that are up-regulated in a cancerous breast, or they may include one or more proteins that are down-regulated in a cancerous breast. Such proteins that are differentially expressed in a cancerous breast versus a non-cancerous breast of the same individual are excellent candidates to be used as bio markers of cancer both in the breast and in other tissues. As such, the present disclosure includes a method of identifying a biomarker for breast cancer or other cancers. Such a biomarker is first identified as being up-regulated or down-regulated in a cancerous breast and can be monitored and correlated to various types of cancers that are more or less aggressive, or more or less invasive. Such a maker, then provides a valuable diagnostic and prognostic tool both prior to and during a therapy regimen.

In addition, certain protein spots that have been identified in a cancerous breast, but not in a non-cancerous breast of the same individual have also been identified in a serum protein profile of the same subject. The use of such bio-markers, first identified in breast cancer, that appear in the serum of any subject, but particularly in the serum of a cancer patient are useful markers for the presence of a cancerous condition in tissues other than the breast. The identification and use of such markers is contemplated by the present invention.

The present disclosure also includes a method for evaluating effectiveness of a chemotherapeutic treatment in a breast cancer patient, including a) obtaining breast ductal secretions from each breast of the subject; b) separating the proteins from the ductal secretions of each breast to obtain a separate protein pattern for the ductal secretions of each breast; c) detecting differences in the protein pattern of one of the breasts compared to the protein pattern of the other breast of the same subject; wherein a significant difference in the protein pattern of the two breasts is indicative of the presence of a neoplasm in one of the breasts; and d) repeating steps a)-c) at timed intervals during the chemotherapeutic treatment of the patient wherein a change in the differences between the protein patterns of the two breasts over time is indicative of an effective chemotherapeutic treatment.

This method may also further include identifying one or more proteins that respond to a chemotherapeutic agent, thereby indicating the course of treatment, and by monitoring changes in the quantity or quality of the protein in a subject after administration of the agent. It is understood that some of the proteins so identified are novel drug targets for which new drugs can be designed constituting new drug discovery.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right. The differences in the protein profiles of the two breasts of this patient are subtle, involving mainly minor protein species only slightly evident upon direct visual inspection.

FIG. 1B demonstrates the electronic analysis of the patterns shown in FIG. 1A and indicates the total number of spots and the location and number of unique spots found in the two samples. The analysis was done with Bio-Rad PDQUEST image analysis software.

FIG. 2A is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right. The differences in this patient's protein profiles are dramatic and readily detectable upon visual inspection.

FIG. 2B demonstrates the electronic analysis of the patterns shown in FIG. 2A and indicates the total number of spots and the location and number of unique spots found in the two samples.

FIG. 2C is a magnification of a portion of the data shown in FIG. 2B.

FIG. 3A is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right. The differences in the two protein profiles for this patient are less dramatic than those shown in FIG. 2, but are still detectable by visual inspection.

FIG. 3B demonstrates the electronic analysis of the patterns shown in FIG. 2A and indicates the total number of spots and the location and number of unique spots found in the two samples.

FIG. 3C is a magnification of a portion of the data shown in FIG. 3B.

FIG. 4 is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right.

FIG. 5 is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right.

FIG. 6 is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right.

FIG. 7 is a side by side comparison of two-dimensional polyacrylamide gel electrophoretic patterns of proteins of nipple aspirate fluid from both of the breasts of a unilateral breast cancer patient. The pattern from the cancerous breast is shown on the left, and the pattern from the normal breast is shown on the right.

FIG. 8A is a side by side comparison of two-dimensional gel electrophoretic patterns of proteins of nipple aspirate fluid from both of the breast of a normal individual who had a negative mammogram within the year prior to providing this sample. The patterns are essentially identical as noted by visual inspection.

FIG. 8B demonstrates the electronic analysis of the protein profiles shown in FIG. 8A. The image analysis software revealed that very few differences exist in the two protein profiles.

FIG. 8C is a magnification of a portion of the data shown in FIG. 8B.

FIG. 9 is a side by side comparison of two-dimensional gel electrophoretic patterns of proteins of nipple aspirate fluid from both of the breast of a normal individual who had a negative mammogram within a year prior to providing the samples. The patterns are determined to be essentially identical by visual inspection.

FIG. 10 is a side by side comparison of two-dimensional gel electrophoretic patterns of proteins of nipple aspirate fluid from both of the breast of a normal individual who had a negative mammogram within a year prior to providing the samples. The patterns are determined to be essentially identical by visual inspection.

FIG. 11 is a side by side comparison of two-dimensional gel electrophoretic patterns of proteins of nipple aspirate fluid from both of the breast of a normal individual who had a negative mammogram within a year prior to providing the samples but who has an abnormal developmental history in that her mother was given the hormone diethylstilbestrol during pregnancy, causing developmental and hormonal abnormalities in the fetus. This resulted in an inability of the individual to conceive. The figure shows that there were very low quantities of nipple aspirate proteins obtained and the sensitive fluorescent stain revealed abnormal patterns. However, the two patterns are essentially identical upon visual inspection. This is a demonstration of the advantage of a patient acting as her own control in testing for unilateral breast cancer in an abnormal hormonal environment.

FIG. 12 is a side by side comparison of two-dimensional gel electrophoretic patterns of proteins of nipple aspirate fluid from both of the breast of an individual (negative mammogram within the last year) whose mother died from breast cancer at the same age as the individual when giving the samples. Even though there are no symptoms, there are differences in the protein patterns detectable upon visual inspection. This difference indicates that more extensive, diagnostic mammogram is recommended.

FIG. 13 is a side by side comparison of two-dimensional gel electrophoretic patterns of proteins of nipple aspirate fluid from both of the breasts of an individual (negative mammogram within the last year) whose family has a profound history of breast cancer. Her maternal grandmother and all her maternal great aunts, a total of four, all had breast cancer, two at 65, two at 50 (similar to individual), and whose mother, the eldest of three female siblings also had breast cancer at 70. The patterns of her nipple aspirate proteins are evidently different upon visual inspection. Furthermore, pI and molecular weight analysis of the spots indicate the presence of all of the known breast cancer markers in the left breast pattern, and not in the right breast pattern. This is a strong indication of the need for further diagnosis including a diagnostic mammogram.

FIG. 14 demonstrates the reproducibility of the quantitative analysis in the methods disclosed herein. This data was obtained by running the same sample on 9 separate gels and the statistics show a relative standard deviation (% CV) of <20%, independent of the protein concentration in the spot. This level of confidence is acceptable for a diagnostic test.

### DETAILED DESCRIPTION

The present disclosure provides a sensitive method for early detection, diagnosis and monitoring of breast cancer by comparing the protein profiles of the nipple aspirate from each breast of an individual and detecting differences in those profiles that are indicative of the presence of a neoplastic state in one or both breasts. By comparing the protein profiles even of normal breasts of an individual over time, one may also obtain information about the hormonal state of the individual and hormonal responses in the breast.

The cells of the lobular and ductal regions of the breast secrete proteins directly into the ducts and these proteins are collected in the nipple aspirate fluid. This fluid is thus a good source of information about the proteins secreted by these cells as analyzed in the 2D gel electrophoresis. Without limiting the present disclosure to any particular theory, it is contemplated that the patterns of proteins secreted by these cells will vary from one individual to another as a function of hormonal stimulus, such as stages of menstrual cycle, pre- or post-menopausal status, oral contraceptive use, hormone replacement therapy, or other factors. For this reason, the comparison between the breasts of an individual provides an almost ideal control, as seen in the markedly similar protein patterns derived from the aspirate of both breasts of the normal volunteers described herein. Even genetically identical tissue (from a twin, for example) would be expected to have differences in protein profile due to the individual hormonal environments.

The lobular and ductal regions are also the place where breast cancer begins. Therefore, it is probable that the pattern of proteins secreted by these cancer cells into the ducts reflects the disease at an early stage. The analysis presented in the present disclosur reflects both of the phenomena, the largely symmetrical hormonal effects, as well as the effects of the localized cancer, in one breast but not in the other. By using the patient as her own control, these two effects can be distinguished. In that case, a threshold number of qualitative differences between the breasts of each individual has the potential to signal the presence of early stage breast cancer. It is also likely that the presence of cancer in both breasts will still lead to a greater than normal difference in the protein patterns from each breast, inasmuch as the degree and stage of cancer in each breast is unlikely to be so symmetrical as to produce identical secretions into both breast ducts.

Among the proteins secreted by the cancerous cells are diagnostic markers for cancer, some of which may well be or reflect the presence of certain drug targets, which conceivably can be used for new drug discovery and or to determine treatment options for the individual patient. The analysis shown in the present study involves the use of highly sensitive staining techniques, capable of detecting proteins in the picogram range. Thus very subtle differences emerge, which could signal the presence of, or potential for cancer at very early stages, perhaps prior to the stage at which cancer is detectable by radiological techniques. The ability to detect and to provide personalized chemotherapeutic regimens for very early stage disease such as pre-cancerous lesions or ductal carcinoma in situ, DCIS, is a very important advance in the treatment of breast cancer.

In preferred embodiments, the protein profiles are obtained by subjecting nipple aspirate fluid to 2D gel electrophoresis. In certain embodiments the first dimensional gel is an isoelectric focusing gel, and the second gel is a denaturing polyacrylamide gradient gel. In certain embodiments the samples may also be subjected to various techniques known in the art for separating proteins. Such techniques include, but are not limited to gel filtration chromatography, ion exchange chromatography, reverse phase chromatography, affinity chromatography, typically in an HPLC or FPLC apparatus, or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectromety or tandem mass spectrometry, or any protein separation technique that determines the pattern of proteins in the mixture either as a one-dimensional, two-dimensional, three-dimensional or multi-dimensional pattern or list of proteins present, or list of their post synthetic modification isoforms.

Proteins are amphoteric, containing both positive and negative charges, and like all the ampholytes, they have the property that their charge depends on the pH. At low pH proteins are positively charged, and at high pH, they are negatively charged. For every protein there is a pH at which they are uncharged, and this is called the isoelectric point. When a charged molecule is placed in an electric field it will migrate towards its opposite charge. In a pH gradient such as those used in the present disclosure, a protein will migrate to the point at which is reaches its isoelectric point and becomes uncharged. The uncharged protein will not migrate further through the gradient and stops. Each protein will stop at its individual isoelectric point and the proteins are thus separated according to charge. In order to achieve optimal separation of proteins, various pH gradients may be used. In the practice of the present disclosed methods, for example, a very broad range of pH, of from about pH 3 to about 11 or about 3-10 may be used, or a more narrow range, such as from about pH 4 to 7 or even pH 7-10 or pH 6-11 may be used as appropriate. The choice of pH range is often determined empirically and such determinations are well within the skill of a practitioner and can be accomplished with no undue experimentation.

In the second dimension, the proteins are separated according to molecular weight by measuring mobility through a polyacrylamide gradient in the detergent SDS. In the presence of SDS and a reducing agent such as DTT, the proteins act as though they are of uniform shape with the same charge to mass ratio. The proteins are then separated by molecular weight on the gel. It is well known in the art that various concentration gradients of acrylamide may be used for such protein separations. For example, a gradient of from about 5% to 20% may be used in certain embodiments, or any appropriate gradient may be used that achieves a satisfactory separation of the proteins in a sample. Other gradients would include, but are not limited to from about 5-18%, 6-20%, 8-20%, 8-18%, 8-16%, 10-16%, or any range as determined empirically.

In certain embodiments the aspirate fluid may be subjected to pre-fractionation protocols such as preparative isoelectric focusing, for example using any one of a number of devices such as a Rotofor (Bio-Rad Laboratories) and commercially available ampholytes, and/or subjected to precipitation by any of a number of reagents alone or in combination such as ammonium sulfate, trichloroacetic acid, perchloric acid, acetone, ethanol, commercial precipitant cocktails such as PlusOne (Amersham Biosciences), or Perfect-Focus (Geno Technology Inc). Additionally, the samples may be subjected to any of immuno-precipitants, affinity capture using solid phase media such as anti-phosphotyrosine antibodies, other antibodies, lectins, attached to chromatography media such as agarose, or any other method designed to separate proteins from a solution into groups or from contaminants such as lipids, nucleic acids, carbohydrates, salts or other substances not required for or interfering in the testing.

In certain embodiments the sample collection and storage may be performed as follows. Immediately upon collection from the patient or volunteer, using any of a number of commercially available nipple aspiration sampling devices, the nipple aspirate fluid is diluted by 100-500 μl of ice cold RPMI buffer, or Tris Buffered Isotonic Saline, or any other appropriate buffer solution, containing a mixture of any of a number of protease inhibitors, for example, PMSF, Leupeptin, Pepstatin, Chyrnostatin, Calpain Inhibitor I®, or Calpain Inhibitor II®, or EDTA-free® protease inhibitor cocktail. The ice cold diluted nipple aspirate fluid is aliquoted into 1.5 ml microfuge tubes in 100 μl portions and frozen in liquid nitrogen.

In certain embodiments, for sample preparation and cleanup, diluted nipple aspirate fluid (100 μl) is suspended in an equal volume of buffer containing 7M urea, 2M thiourea, 1% triton X-100, 1% CHAPS®, 1% DTT and 1% Ampholyte® pH 3-10 (Bio-Rad or another suitable vendor). Alternatively, the aspirate fluid may be suspended in high concentration urea, Nonidet P-40, CHAPS (SIGMA, ST. Louis, Mo.) and Resolyte or other ampholytic media. Initial precipitation is performed using Perfect-Focus® (Geno Technology Inc). For each 100 μl of above sample mixture, 300 μl of ice cold dilute aqueous TCA (˜10%) is added, mixed and incubated on ice for 15 minutes. Then 300 μl of ice cold dilute NaOH is added and the mixture vortexed and centrifuged cold at 15,000×g for 5 minutes. The supernatant is removed by careful aspiration and the tube is centrifuged again. The remaining supernatant is removed. For follow-on precipitation, ultra pure water, 25 μl or an equal volume to the pellet, is then added to the tube, vortexed for 30 seconds and 1 ml of −20° C. acetone and 4 μl cellulose suspension is added to the pellet. The pellet is then fully suspended by vortexing and incubated at −20° C. for 30 minutes with periodic vortexing. The suspension is then centrifuged at 15,000×g for 10 minutes and the supernatant is immediately removed carefully by vacuum aspiration. The tubes are then placed open in a speed vacuum centrifuge for 1 minute to remove volatile organic solvent, without completely drying the pellet. The follow-on precipitation may also be repeated. Protein concentration is measured preferably using the RC DC® assay kit (Bio-Rad Laboratories).

In certain embodiments the 2D gel protein data may be obtained as follows. An aliquot of 80 μg of protein is loaded into an 11 cm IEF strip, pH 4-7. Focusing is conducted on IEF cells at 250 V for 20 minutes followed by a linear increase to 8000 V for 2½ hours. The focusing is terminated at 20,000 V-hr. Strips are then equilibrated in 375 nM Tris buffer, pH 8.8, containing 6 M urea, 20% glycerol, 1% DTT, and 2% SDS. Fresh DTT is added to the buffer at a concentration of 30 mg/ml for 15 minutes, followed by an additional 15 minutes in the same buffer containing freshly added iodoacetamide (40 mg/ml). Strips are then loaded onto the second dimension using Bio-Rad Criterion gradient gels with an 8-16% acrylamide gradient. Gels are then preferably stained by SpyroRuby fluorescent dye. Other dyes such as silver staining and coomassie blue are also known in the art and may be used in certain embodiments, but the fluorescent dyes are more sensitive and are preferred in most cases. Gel images are compared visually and/or electronically using PDQUEST® software (Bio-Rad Laboratories). Analysis includes spot detection and comparisons of protein patterns from both breasts using internal protein standards as landmarks.

It is an important aspect of the present disclosure that the protein spots that are identified as differentially expressed in a cancerous tissue may isolated, identified and that antibodies to such proteins may be available. The production of an antibody to an isolated protein is well known and routine in the field and is not described in detail herein.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the cancer bio-markers identified by the present methods may be used in a variety of assays in order to obtain a protein profile from a nipple aspirate, serum or other fluid or tissue sample. Well known methods include immunoprecipitation, antibody sandwich assays, ELISA and affinity chromatography methods that include antibodies bound to a solid support. Such methods also include microarrays of antibodies or proteins contained on a glass slide or a silicon chip, for example.

It is contemplated that arrays of antibodies to cancer markers known and identified by the present methods, or the protein markers themselves may be produced in an array and contacted with the serum or ductal secretion samples described herein or with the antibodies as appropriate in order to obtain a protein profile. The use of such microarrays is well known in the art and is described, for example in U.S. Pat. No. 5,143,854, incorporated herein by reference.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Protein samples were obtained from female patients, each with cancer in one breast, the other breast normal, and from healthy female volunteers with both breasts normal. From each patient and the volunteers, one sample was aspirated from each breast and a serum sample was also obtained. Protein spots were detected when the samples were subjected to two-dimensional polyacrylamide gel electrophoresis using an iso-electric focusing (IEF) strip of pH 4-7 for the first dimension and a sodium-dodecyl-sulfate (SDS) polyacrylamide gel for the second dimension with an 8-16% polyacrylamide gradient (Bio-Rad), followed by fluorescent staining with Spyro Ruby Red® and image analysis. When the images of the 2D gels of the three samples from each patient were compared to each other using PDQUEST® image analysis software, definite qualitative differences (PlaX™) were observed between the protein patterns in the samples from each breast. In addition, some of these differences were reflected in the protein patterns in the serum of the subjects.

Comparisons of the differences between the breast aspirates of the patients indicated that the differences varied in magnitude from relatively limited differences involving proteins found only in minor amounts to marked differences that were obvious even among abundant protein spots. In contrast, the protein patterns obtained with the samples from each breast of the normal volunteers were essentially identical with no qualitative differences found between the aspirates from both breasts. This was evident among the most and least abundant proteins observed. Inasmuch as the differences observed in the present example are between nipple aspirate protein patterns from cancerous and non-cancerous breasts of individual patients, i.e. where the patient is acting as her own control, the most likely presumption is that some of the differences observed in this example reflect elements of the fine structure of the disease. The patient-to-patient variability of these differences may well reflect variables in the disease fine structure, which are potentially of clinical diagnostic significance to the individual patients. In conclusion, proteomic analysis of breast nipple aspirate samples, as disclosed herein, is contemplated to be a useful diagnostic tool for breast cancer detection, prognosis, and follow-up during therapy.

The number of spots detected by PDQuest® image analysis of SpyroRuby Red® stained two-dimensional gels (pH 4-7) obtained upon electrophoresis of 80 μg of nipple aspirate fluid proteins are listed in Table 1. Shown are the results from six different individuals with cancer in one breast and the other breast non-cancerous, and one normal individual with both breasts non-cancerous. In the case of the cancer patients, there were substantial differences between the protein patterns in their cancerous and non-cancerous breasts. For example, the number of protein spots detected in the cancerous breast and not detected in the non-cancerous breast from the same patient varied from 30-202 spots. In addition, the number of protein spots detected in the non-cancerous breast and not detected in the cancerous breast of the same patient varied from 14 to 73 spots. On the other hand, in the case of the normal individual, the number of protein spots detected in the left breast and not detected in the right breast and vice versa was only 3 and 2 respectively.

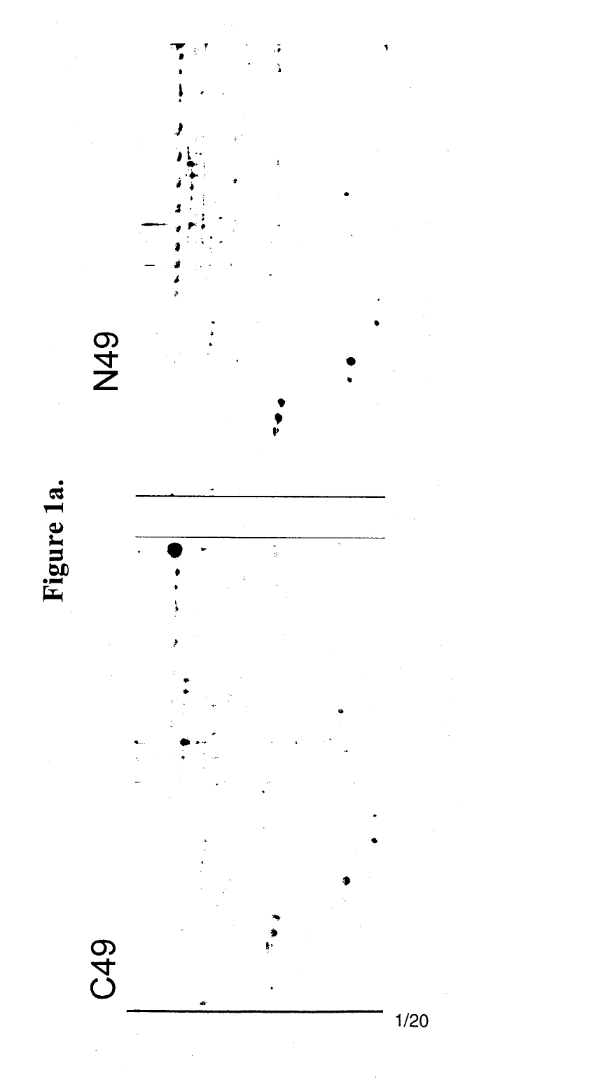

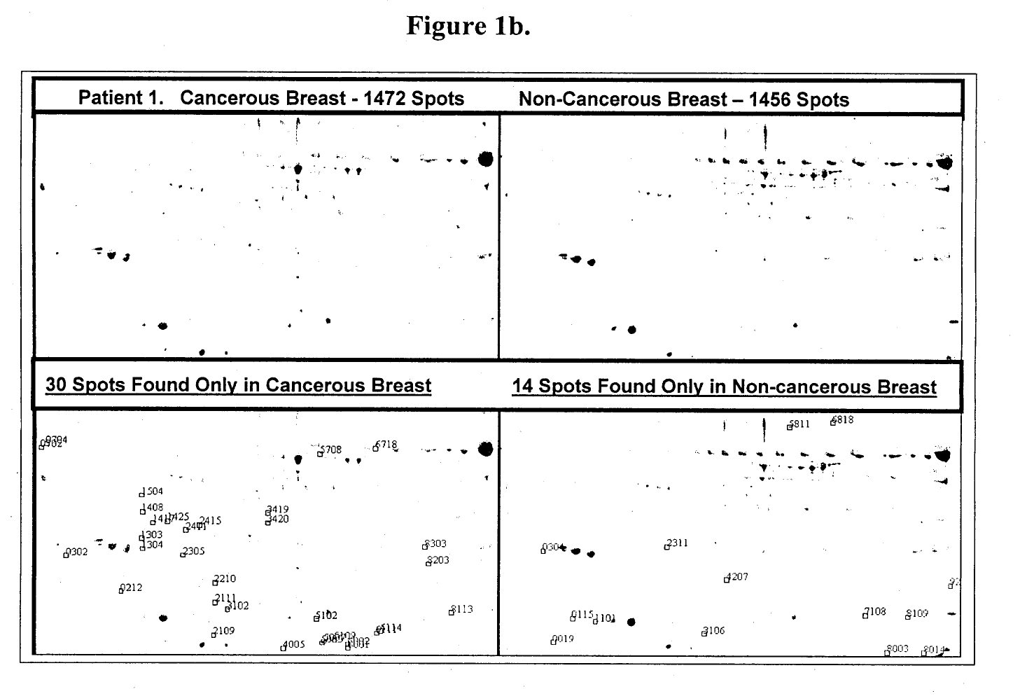

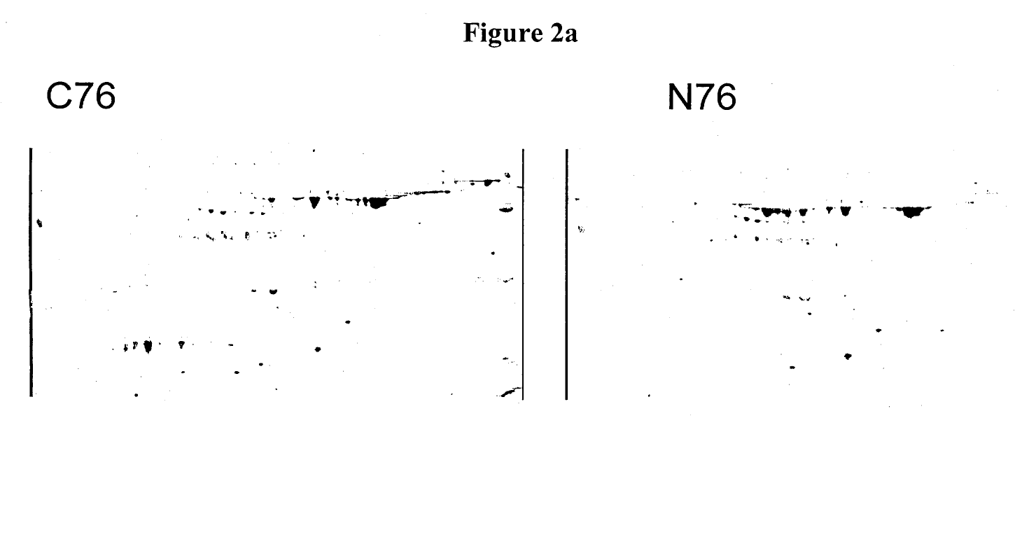

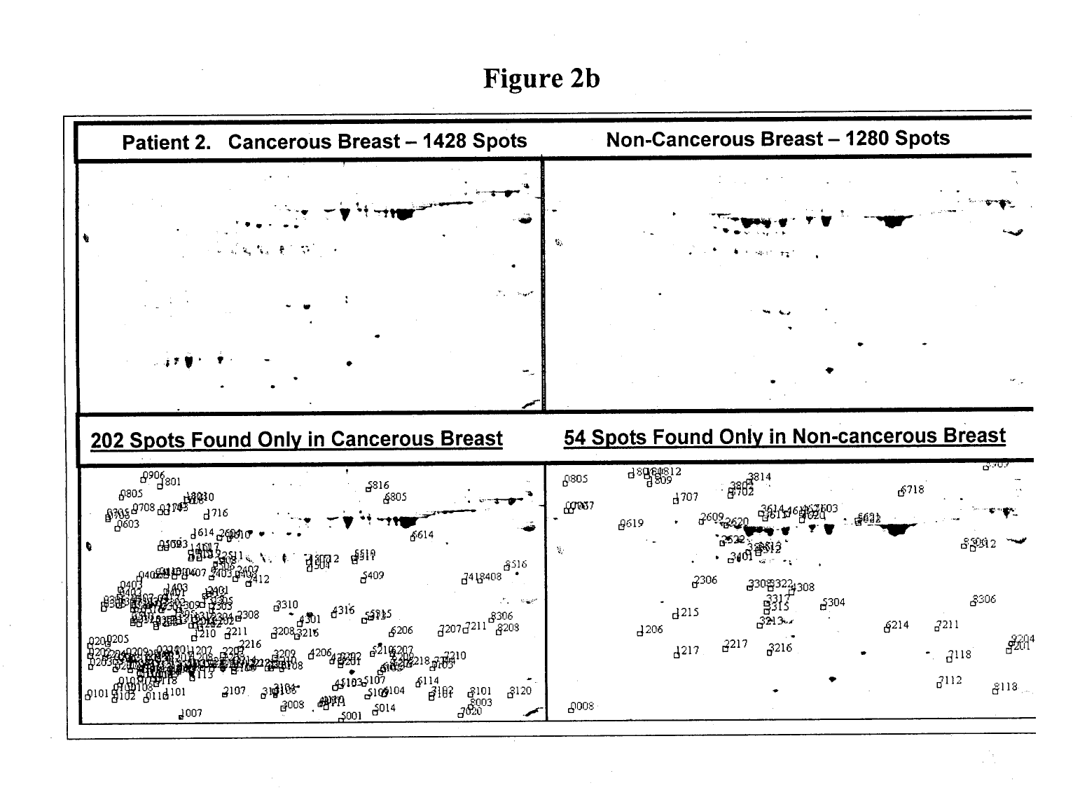

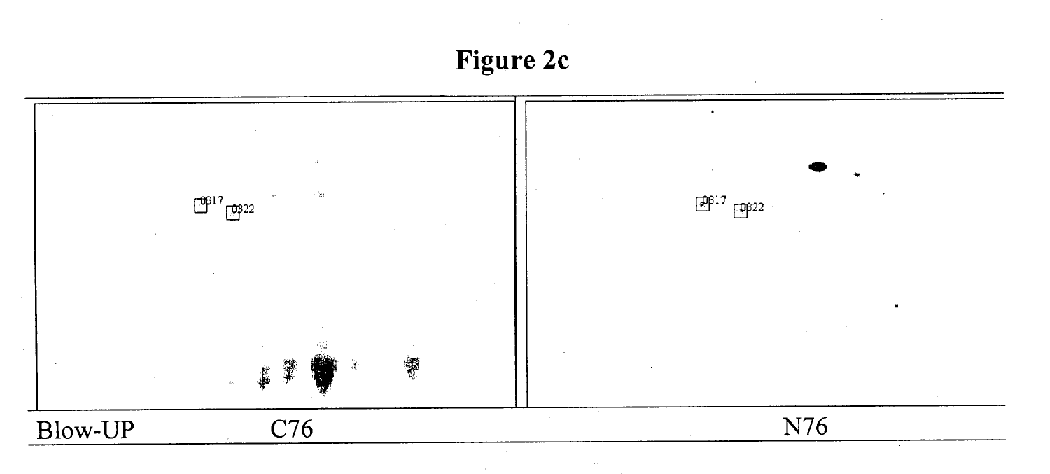

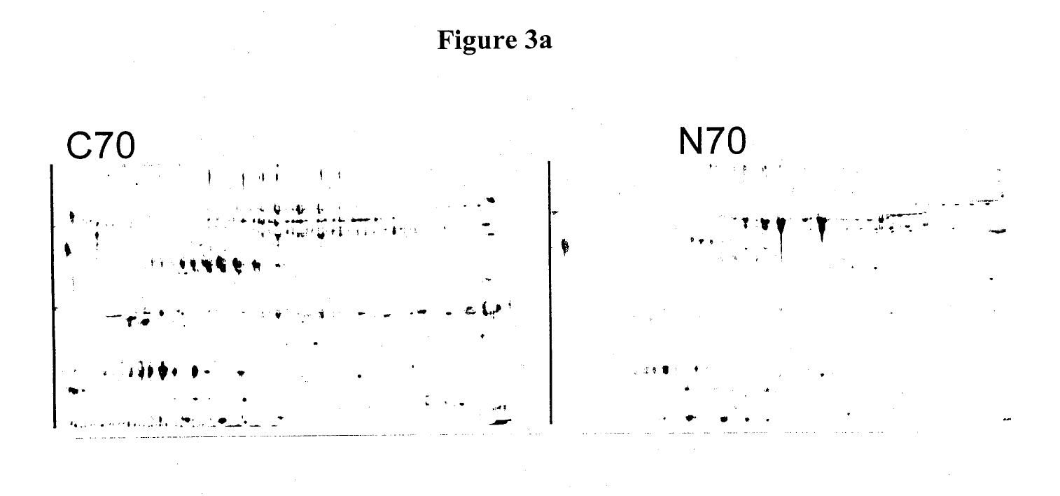

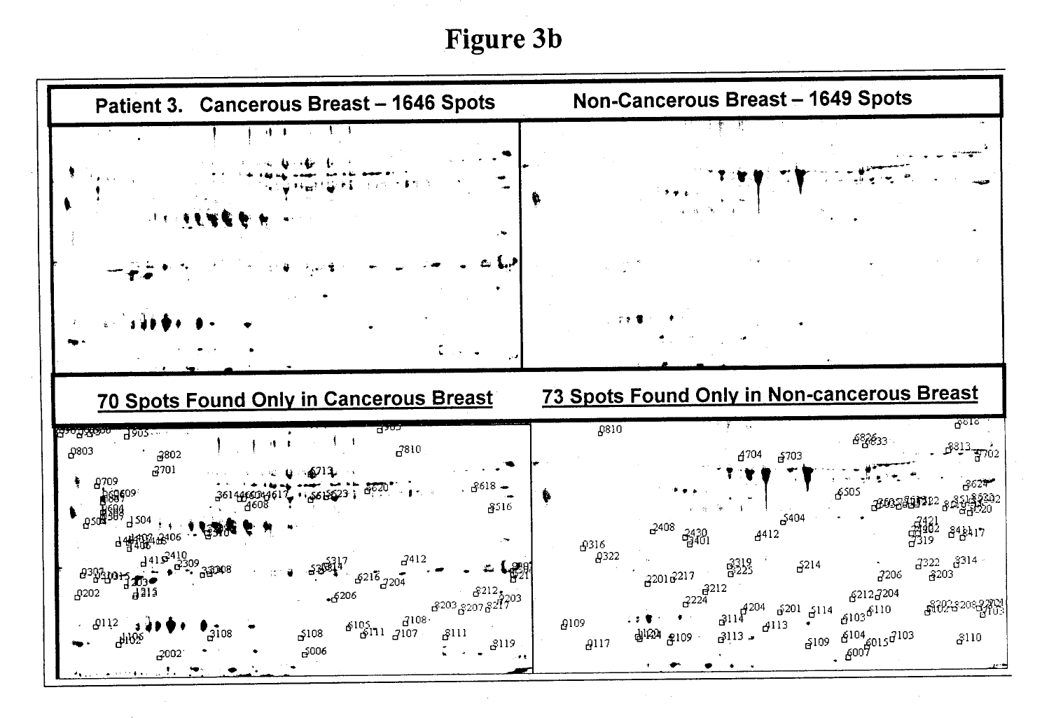

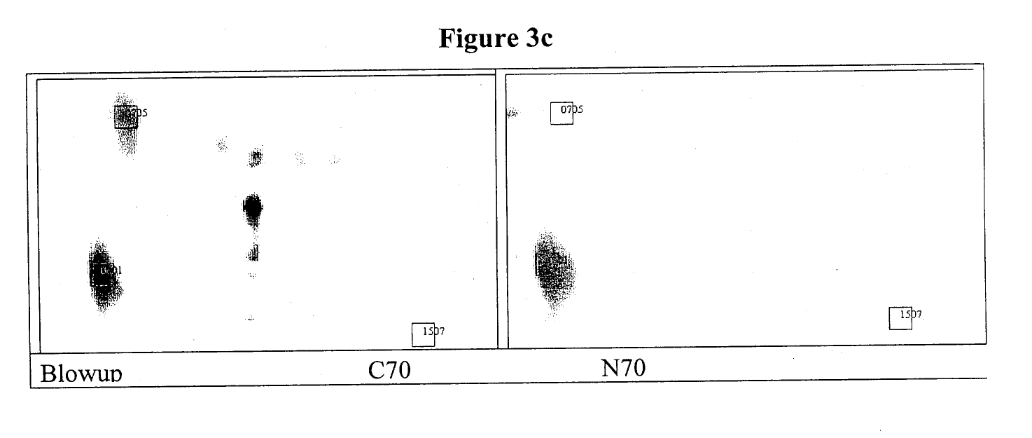

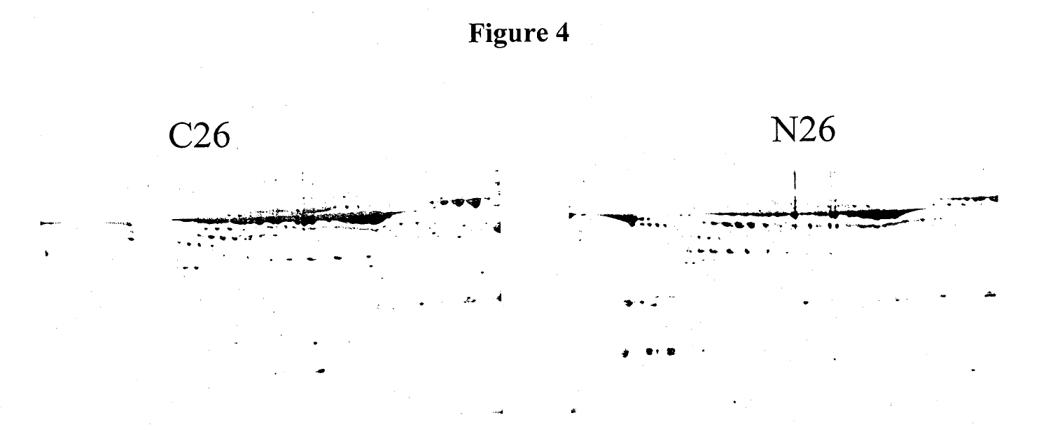

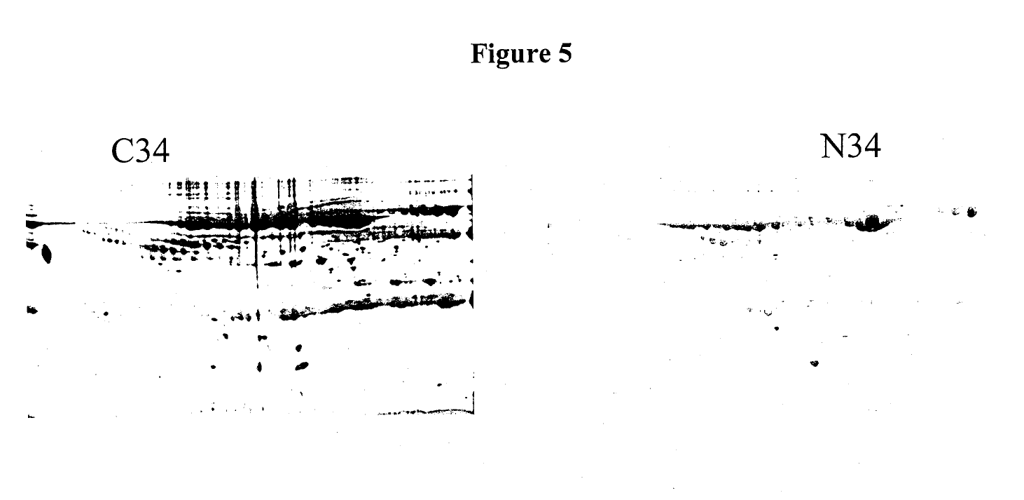

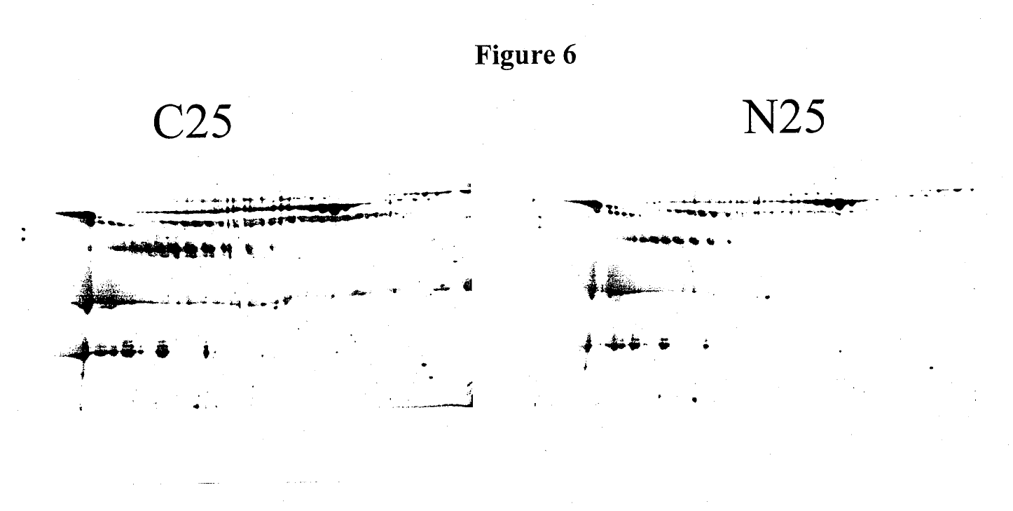

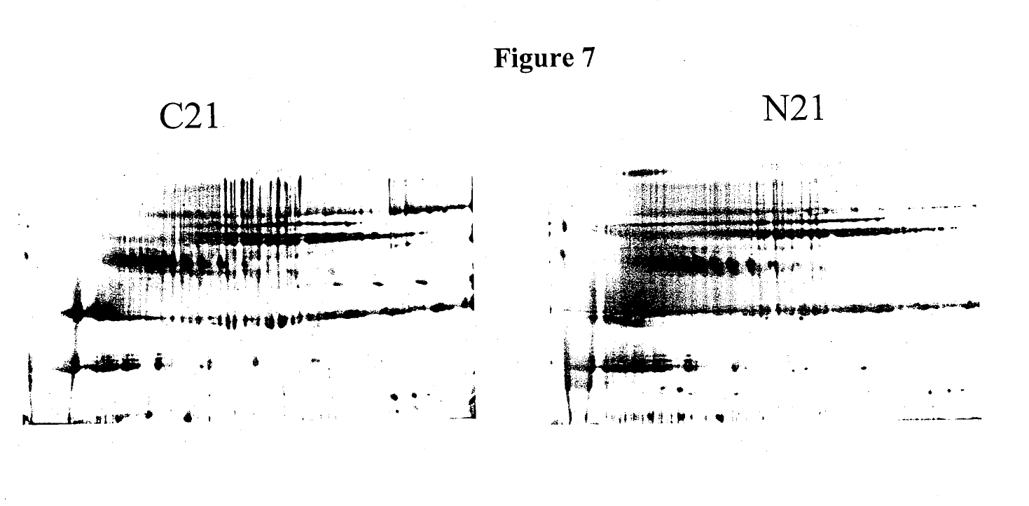

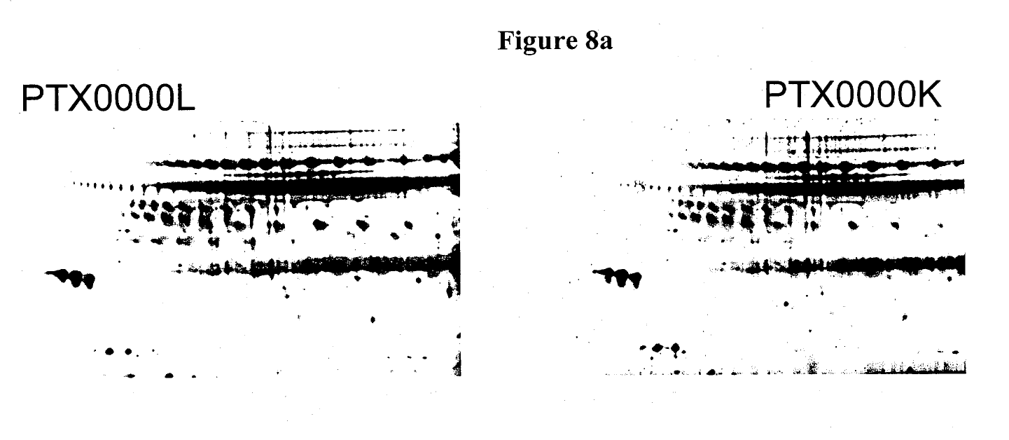

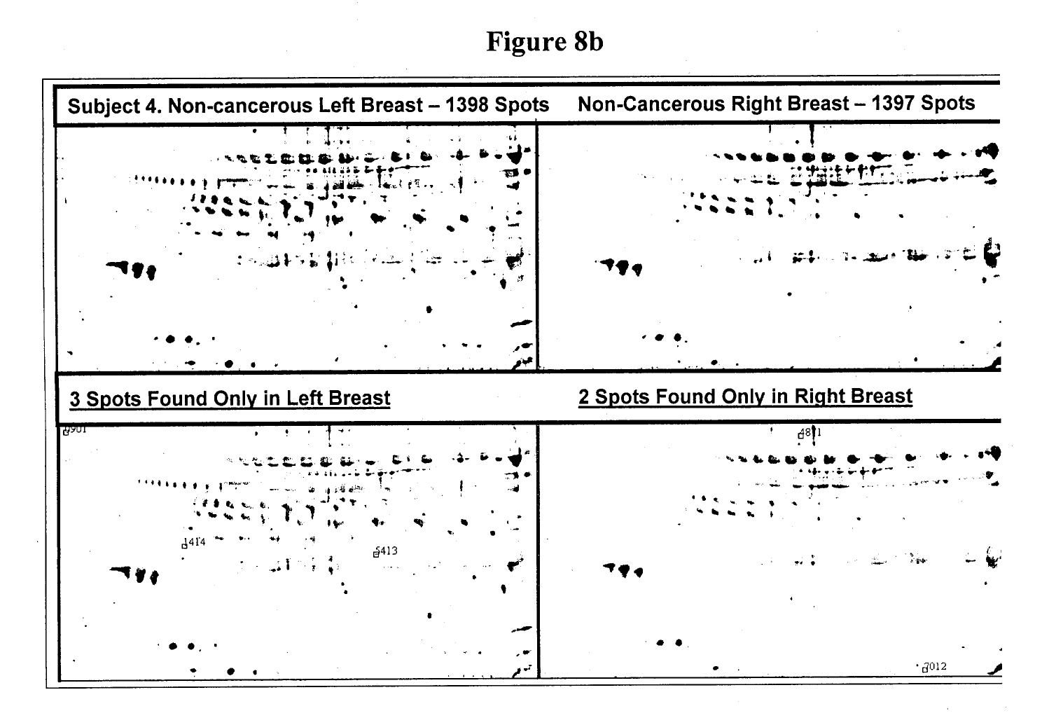

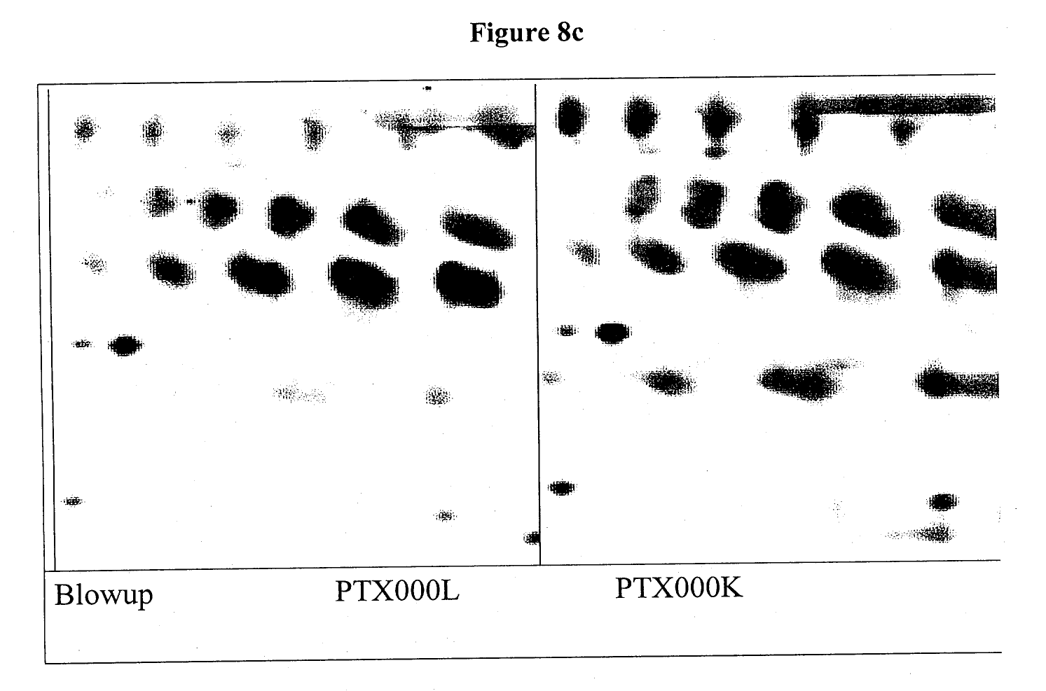

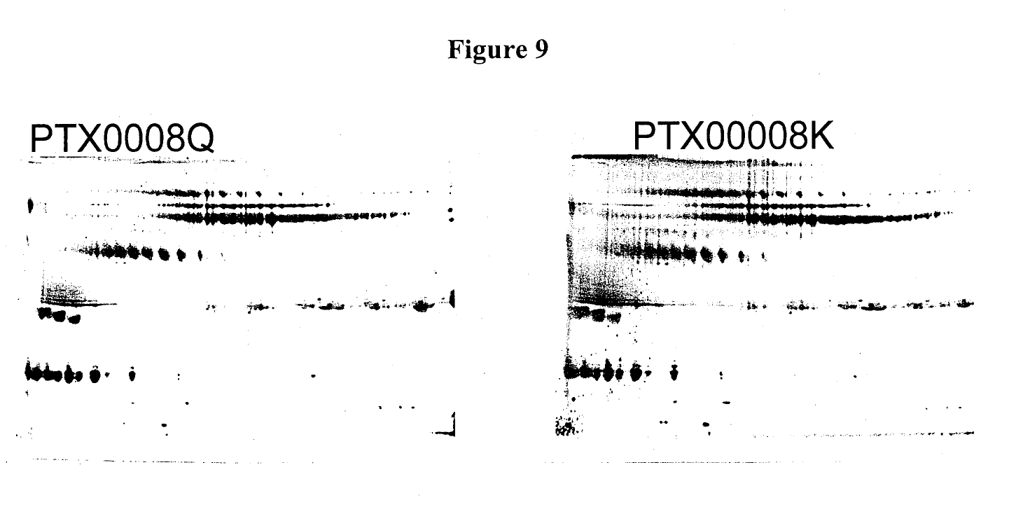

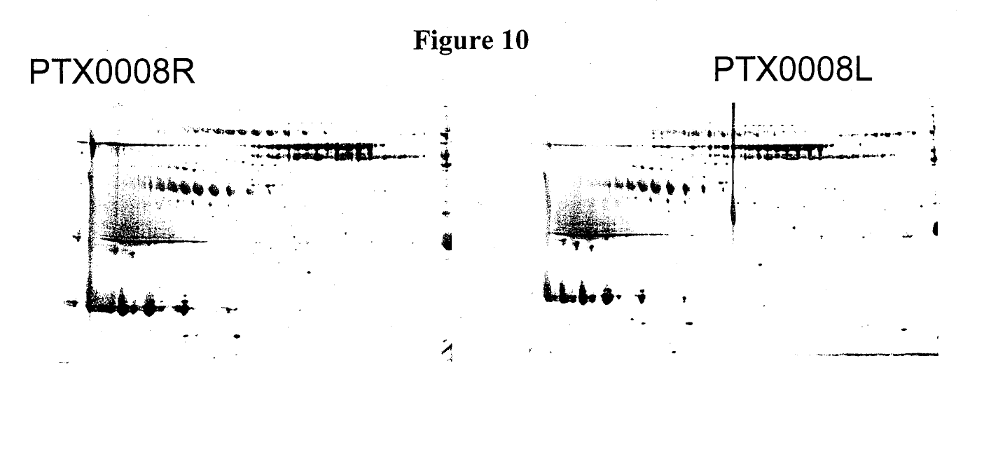

These qualitative differences in protein spot patterns are detailed in the attached drawings. In the cancer patients (FIGS. 1-7), the spots that were detected in one breast and not in the other varied in molecular weight (vertical position), isoelectric point (horizontal position), and abundance (spot intensity), and these factors were different from patient to patient. For example, with patient 1 (FIG. 1), the differences were much more subtle than in the case of patient 2 (FIG. 2). The differences found with patient 3 (FIG. 3) were of intermediate degree, with somewhat more differences in proteins detected in the non-cancerous breast in that patient vs. the previous two. Most strikingly, the patterns of proteins from the right and left breasts of the normal individuals (FIGS. 8-10) were almost identical qualitatively.

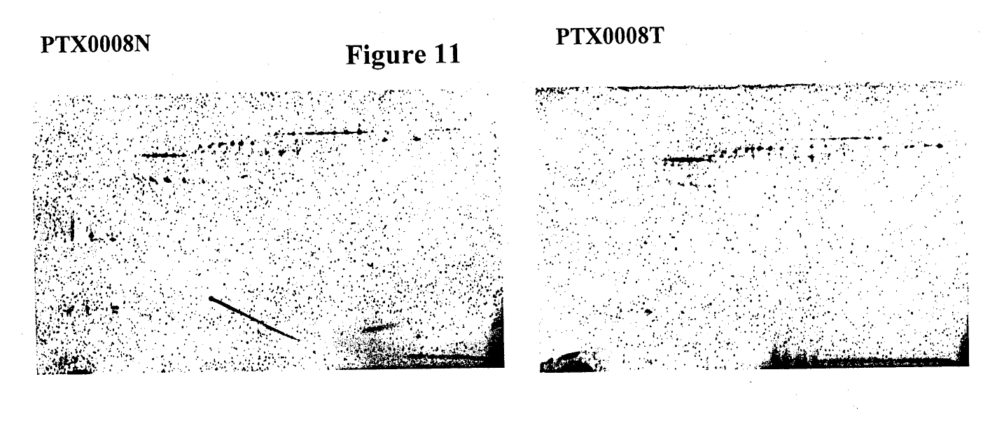

The methods disclosed herein are also demonstrated to be efficacious in routinely or annual screening of subjects who may be at higher risk of breast cancer than the population at large. The subjects shown in FIGS. 11-13 all have either an abnormal hormonal profile, or have a family history of breast cancer. The data shown in these figures demonstrate that the methods are beneficial in early detection and screening of such subjects.

For example, the subject whose protein profiles are shown in FIG. 11 has an abnormal developmental history because her mother was given DES, which has an estrogen like activity during pregnancy. There were very low quantities of nipple aspirate proteins obtained from this subject, below the detectable level in the normal protein assays practiced by the inventors to determine protein levels in the nipple aspirates. In spite of her unusual hormonal profile, the present methods were able to determine upon visual inspection, that there was essentially little or no difference in the protein profiles of her two breasts.

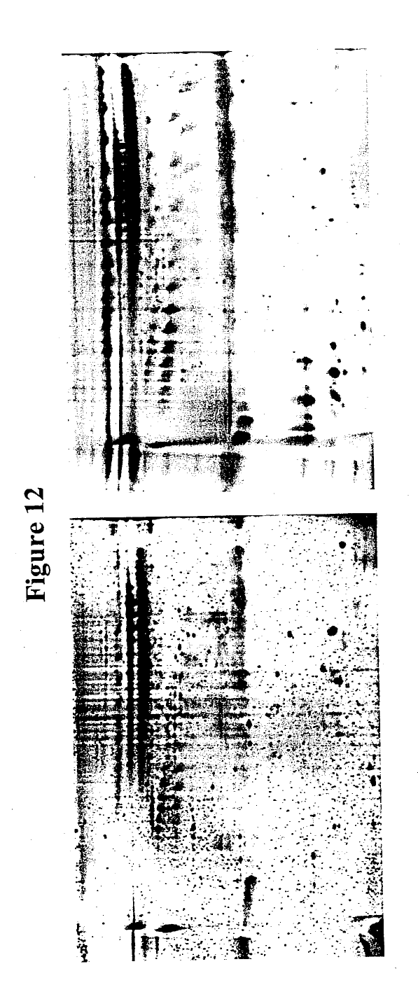

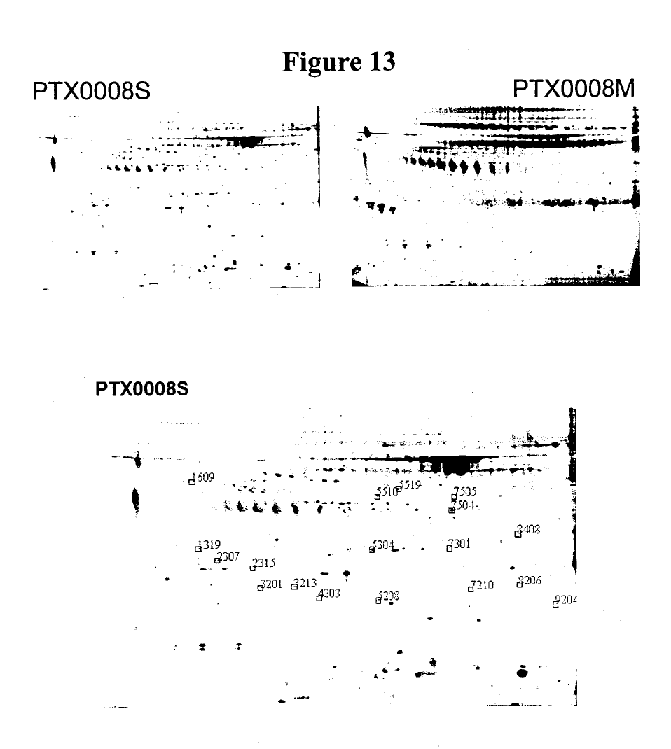

The present methods are also shown to be efficacious in screening of subjects with a higher risk of breast cancer based on family history. FIG. 12 is the protein profiles of the two breasts of a subject whose mother died of breast cancer, and who had a negative mammogram. Although the quantitative analysis of the profiles is not shown here, visual inspection of the profiles indicates that they are sufficiently different to warrant further more intense diagnostic procedures to possibly detect an early stage condition. Another example of a protein profile of a higher risk individual is shown in FIG. 13. This subject has a profound history of breast cancer in her family, and had a negative mammogram within the year prior to providing these samples. As shown in the figure, the protein profiles of her two breasts are quite different as determined by visual inspection. In addition, many of the protein spots that appear only in the left breast were analyzed for pI and molecular weight. Based on these parameters, all known breast cancer markers appeared in the profile of this breast. Again, in spite of the negative mammogram, the present methods have uncovered reason for concern at a much earlier stage than has previously been possible. The detected cancer markers are shown in Table 2.

The lack of detection of a protein spot can be due to its absence or to its down-regulation to below the level of detection of the stain. Similarly, the detection of a protein spot can also be due to its induction or to up-regulation to amounts above the level of detection of the stain. In fact, many of the protein differences noted in this analysis involved proteins found in relatively low abundance, where detection limit may well have played a significant role. In other words, depending on the detection limit of the stain used, a qualitative difference may actually be a quantitative difference at a lower detection level. All such methods that detect a difference in protein pattern, either quantitative or qualitative, would, however, fall within the scope of the present disclosure.

TABLE 1
Qualitative comparison of the number of spots detected in 2D gel
electrophoretic protein patterns of nipple aspirates from the cancerous and non-cancerous
breast of six individual patients and from both non-cancerous breasts of an individual
normal subject.

| | Protein Spots Only in | Protein Spots Only in | |
|---|---|---|---|
| | Cancerous | non-Cancerous | Total Number of |
| | Breast/Total Detected | Breast/Total Detected | Difference |
| | in the Cancerous | in the Non-cancerous | Proteins Between |
| Sample Source | Breast | Breast | the Two Breasts |
| Cancer Patient 1 | 30/1472 | 14/1456 | 44 |
| (FIG. 1) | | | |
| Cancer Patient 2 | 202/1428 | 54/1280 | 256 |
| (FIG. 2) | | | |
| Cancer Patient 3 | 70/1646 | 73/1649 | 143 |
| (FIG. 3) | | | |
| Cancer Patient 4 | 31 | 45 | 76 |
| Cancer Patient 5 | 81 | 152 | 233 |
| Cancer Patient 6 | 33 | 14 | 47 |
| Cancer Patients 95% | | | 17.4 |
| Confidence Lower | | | |
| Limit | | | |
| | Number of Protein | Number of Protein | |
| | Spots Only/Total | Spots Only/Total | |
| | Detected in the Non- | Detected in the Non- | |
| | cancerous Left Breast | cancerous Right | |
| | of the Same Normal | Breast of the Same | |
| | Subject | Normal Subject | |
| Normal Subject 4 | 3/1398 | 2/1397 | 5 |
| (FIG. 8) | | | |

TABLE 2
Known breast cancer markers found in the left breast but not in the
right breast of an individual with negative mammogram but profound
medical history of breast cancer in her family. The 2D gel data is shown
in FIG. 13.

| Protein Marker | Mass (kDa)/pI | SSP on gel | Mr/pI on gel |
|---|---|---|---|
| 14-3-3 alpha/beta | 27.7/4.79 | 3209 | 27.7/4.9 |
| 14-3-3 sigma | 27.7/4.65 | 3201 | 27.3/4.8 |
| 14-3-3 zeta/delta | 27.7/4.78 | 3213 | 27.5/4.3 |
| Annexin I | 38.8/6.6 | 8408 | 38.5/6.5 |
| Annexin III | 33.6/5.63 | 7301 | 36.6/5.9 |
| | | 5304 | 36.4/5.3 |
| Annexin V | 31/4.94 | 2315 | 32.2/4.8 |
| Calreticulin | 55/4.3 | 1609 | 57.8/4.6 |
| Cathepsin D | 43.2/5.9 | 7504 | 41.9/5.9 |
| Cytokeratin-K8 | 53.5/5.52 | 5519 | 52.0/5.5 |
| Cytokeratin-K18 | 44.4/5.3 | 5510 | 46.9/5.3 |
| GST | 24.2/5.6 | 5208 | 24.6/5.3 |
| HER2/neu | 21.3/6.9 | 9204 | 23.9/6.8 |
| HSP-27 | 27.1/5.8-6.6 | 8206 | 27.9/6.5 |
| | | 7210 | 26.9/6.1 |
| Maspin | 42.1/5.72 | 7504 | 41.9/5.9 |
| PCNA | 32/4.57 | 2307 | 34.1/4.7 |
| | | 1319 | 36.6/4.6 |
| PTEN | 47.2/5.9 | 7505 | 46.9/5.9 |
| Rho GDI | 27.6/4.9 | 4203 | 25.1/5.0 |

That many of the differences found in this example involve low abundance proteins is thought to reflect the fact that tumor markers and drug targets often are regulatory in nature, such as members of signal transduction pathways, and are often among the less abundant proteins. The presence of such proteins, bFGF (basic fibroblast growth factor) and VEGF (vascular endothelial growth factor), for example, has been demonstrated in nipple aspirates in low amounts (pg-ng/ml levels) by ELISA, which vary with lactation and, in the case of bFGF, are significantly increased in breast cancer in the nipple aspirates (Liu, et al., The Lancet 356:567 ,2000).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for detecting a neoplasm in the breast of a subject comprising:
obtaining breast ductal secretions from each breast of the subject;
separating the proteins from the ductal secretions of each breast to obtain a separate protein pattern for the ductal secretions of each breast; and
detecting differences in the protein pattern of one of the breasts compared to the protein pattern of the other breast of the same subject;
wherein a difference in the protein pattern of the two breasts is indicative of the presence of a neoplasm in at least one of the breasts.

2. The method of claim 1, wherein the ductal secretions are obtained by nipple aspiration.

3. The method of claim 1, wherein the ductal secretions are obtained by ductal lavage.

4. The method of claim 1, wherein a difference in the total number of proteins detected in the protein patterns is indicative of a neoplasm in at least one of the breasts.

5. The method of claim 1, wherein the presence of known cancer markers in the protein pattern of one of the breasts is indicative of a neoplasm in that breast.

6. The method of claim 1, wherein a difference in quantity of one or more proteins in the protein profiles of the two breasts is indicative of a neoplasm in one of the breasts.

7. The method of claim 1 wherein the neoplasm is cancer.

8. The method of claim 1, wherein the neoplasm is benign.

9. The method of claim 1, wherein the neoplasm is ductal carcinoma in situ.

10. The method of claim 1, wherein the neoplasm is a pre-cancerous condition.

11. The method of claim 1, wherein the protein patterns are obtained by subjected the ductal secretion samples to two dimensional electrophoresis.

12. The method of claim 11, wherein the two dimensional electrophoresis comprises a separation by isoelectric point followed by a separation by molecular weight.

13. The method of claim 11, wherein a difference in the protein pattern of one of the breasts compared to the protein pattern of the other breast of the same subject is detected by visual inspection of the stained proteins.

14. The method of claim 11, wherein a differences in the protein pattern of one of the breasts compared to the protein pattern of the other breast of the same subject is detected by an electronic image analysis program.

15. The method of claim 1, wherein a difference in the protein pattern of one of the breasts compared to the protein pattern of the other breast of the same subject is indicative of neoplasms in both breasts.

16. The method of claim 1, wherein the protein pattern is obtained by contacting the ductal secretions with antibodies to at least a portion of the proteins contained within the ductal secretions.

17. A diagnostic test for female subjects, comprising
obtaining breast ductal secretions from each breast of the subject;
separately subjecting the secretions of each breast to two dimensional gel electrophoresis to obtain a protein pattern for the secretions of each breast; and
comparing the protein pattern obtained from the secretions of the two breasts of the same subject;
wherein a difference in the protein pattern of the two breasts is indicative of the presence of an abnormal condition in at least one of the breasts.

18. The test of claim 17, wherein the abnormal condition is a cancerous condition.

19. The test of claim 17, wherein the abnormal condition is a pre-cancerous condition.

20. The test of claim 17, wherein the abnormal condition is a benign condition.

21. The test of claim 17, wherein the first step of the two dimensional gel electrophoresis is an isoelectric focusing step.

22. The test of claim 21, wherein the iso-electric focusing step comprises use of an iso-electric focusing strip with a range of pH 4-7, pH 7-10, pH 3-10, or pH 6-11.

23. The test of claim 17, wherein the second step of the two dimensional gel electrophoresis is electrophoresis through a sodium-dodecyl-sulfate (SDS) polyacrylamide gel with an 8-16% polyacrylamide gradient.

24. The test of claim 17, wherein the protein patterns are stained.

25. The test of claim 24, wherein the stain is a fluorescent stain or a visible stain.

26. The test of claim 17, wherein the protein patterns are compared visually.

27. The test of claim 17, wherein the protein patterns are digitized and compared electronically.

28. The test of claim 17, wherein the breast ductal secretions are pre-fractionated prior to two dimensional gel electrophoresis.

29. The test of claim 28, wherein the secretions are pre-fractionated by preparative isoelectric focusing, precipitation or affinity capture.

30. The method of claim 28, wherein the proteins are precipitated in ethanol, acetone or ammonium sulfate.

31. A method of identifying a biomarker for breast cancer comprising:
obtaining breast ductal secretions from each breast of a subject in which one breast is normal and one is cancerous;
separating the proteins from the ductal secretions of each breast by two dimensional gel electrophoresis to obtain a separate protein pattern for the ductal secretions of each breast;
detecting proteins that are differentially expressed in the cancerous breast as compared to the normal breast of the same subject; and
identifying a protein that is differentially expressed, wherein the identified protein is a marker of breast cancer.

32. The method of claim 31, wherein the identified protein is expressed at a higher level in a normal breast than in a cancerous breast.

33. The method of claim 31, wherein the identified protein is expressed at a higher level in a cancerous breast than in a normal breast.

34. The method of claim 31, wherein the identified protein is removed from the two dimensional gel by a robotic arm for further analysis.

35. The method of claim 31 wherein the protein is identified by in gel digestion and matrix assisted laser desorption time of flight mass spectrometry (MaldITOF MS) based peptide mass fingerprinting and database searching or liquid chromatography with tandem mass spectrometry sequencing of individual peptides (LCMS/MS).
